(19) United States
(12) Patent Application Publication
(10) Pub. No.: US 2008/0081075 A1
(43) Pub. Date: Apr. 3, 2008

(54) Multifunctional mixed micelle of graft and block copolymers and preparation thereof

(75) Inventors: Ging-Ho Hsiue, Hsinchu (TW); Chun-Liang Lo, Hsinchu (TW); Ko-Min Lin, Hsinchu (TW); Chun-Kai Huang, Hsinchu (TW); Hung-Hao Chen, Hsinchu (TW)

Correspondence Address: BACON & THOMAS, PLLC, 625 SLATERS LANE, FOURTH FLOOR, ALEXANDRIA, VA, 22314, US

(73) Assignee: National Tsing Hua University, Hsinchu (TW)

(21) Appl. No.: 11/657,468

(22) Filed: Jan. 25, 2007

Related U.S. Application Data
(60) Provisional application No. 60/848,382, filed on Oct. 2, 2006.
(60) Provisional application No. 60/848,381, filed on Oct. 2, 2006.

(51) Int. Cl. A61K 9/50; A61K 9/16

(52) U.S. Cl. 424/490

(57) ABSTRACT

The present invention discloses a novel mixed micelle structure with a functional inner core and hydrophilic outer shells self-assembled from a graft macromolecule and one or more block copolymer, and preferably from a graft copolymer and two or more diblock copolymers. The micelle synthesized in the present invention has a size of about 50-200 nm, which can be used as a cancer diagnosis agent and a cancer drug delivery carrier.

### FIELD OF THE INVENTION

The present invention is related to a polymeric micelle having a core-shell structure, and in particular to a multifunctional micelle having a core-shell structure from self-assembly of a graft copolymer and at least one diblock copolymer.

### BACKGROUND OF THE INVENTION

Research on multicomponent micelles for biomedical applications has generally shown that virtually all types and classes of micelles exhibit beneficial properties, such as specific functionality, enhanced specific tumor targeting, stabilized nanostructures, overcame defects from various materials, and displayed multifunctions.^{[1-4]} In the polymer field, multicomponent micelles (also called mixed micelles) have been widely investigated in di-diblock copolymer, di-triblock copolymer, tri-triblock copolymer, and graft-diblock copolymer systems.^{[2-10]} However, no work has yet described the mixed micelle system based on a graft copolymer and a diblock copolymer or several diblock copolymer. Over the last decade, most studies were concerned with micellization theories of mixed micelles.^{[5-10]} However, few studies examine drug delivery.^{[3,4]} Mixed micelles are quite complicated, and the complete core-shell structure cannot be observed clearly. This creates a bottleneck in biomedical applications.

### SUMMARY OF THE INVENTION

The present invention discloses a novel mixed micelle structure with a functional inner core and hydrophilic outer shell self-assembled from a graft macromolecule and one or more block copolymer, and preferably from a graft copolymer and two or more diblock copolymers. The micelle synthesized in the present invention has a size of about 50-200 nm.

The present invention provides a polymeric micelle having a core-shell structure, wherein said structure comprises a graft macromolecule and a block copolymer, said graft macromolecule comprising a backbone and hydrophobic side chains bound to the backbone, said block polymer comprising a hydrophobic polymeric segment and a hydrophilic polymeric segment, wherein the hydrophobic side chains of said graft macromolecule are aggregated, and the hydrophobic polymeric segment of said block polymer is packed and associated to the aggregated hydrophobic side chains of the graft macromolecule with the hydrophilic polymeric segment of the block polymer extruding therefrom to form the core-shell structure.

The present invention further provides a process for preparing a polymeric micelle having a core-shell structure, which comprises the following steps:

a) dissolving a graft macromolecule and a block copolymer in an organic solvent, wherein said graft macromolecule comprises a backbone and hydrophobic side chains bound to the backbone, and said block polymer comprises a hydrophobic polymeric segment and a hydrophilic polymeric segment,

b) subjecting the resulting polymer solution from step a) to a dialysis treatment against water to replace the organic solvent in the solution with water.

Preferably, the process of the present invention further comprises c) freeze-drying the resulting aqueous solution from step b) to obtain dried polymeric micelle.

Preferably, one or more different block copolymers are dissolved in the organic solvent in step a).

Preferably, a drug is dissolved in the organic solvent together with the graft macromolecule and the block copolymer in step a).

Preferably, the hydrophobic side chains and the hydrophobic polymeric segment comprise a same repeating unit.

Preferably, the block copolymer is a diblock copolymer comprising the hydrophobic polymeric segment and the hydrophilic polymeric segment. More preferably, said diblock copolymer is methoxy-poly(ethylene glycol)-b-poly(D,L-lactide).

Preferably, the hydrophobic polymeric segment has a number-average molecular weight of 500-2500, and the hydrophilic polymeric segment has a number-average molecular weight of 2000-10000.

Preferably, the hydrophobic polymeric segment of the block copolymer is bioresorable.

Preferably, the hydrophobic polymer segment of the block copolymer is poly(ester), poly(lactide), poly(lactic acid), or polycaprolactone. More preferably, the hydrophobic polymer segment of the block copolymer is poly(lactide).

Preferably, the hydrophilic polymeric segment of the graft copolymer is polyacrylate, or a pH-/ionic strength sensitive polymer which is a poly(acrylic acid), poly(methacrylic acid), poly(butenedioic acid), polyhistidine or poly(vinyl imidazole).

Preferably, the hydrophilic polymeric segment of the block copolymer is poly(ester), poly(ethylene glycol), methoxy-poly(ethylene glycol), or poly(2-ethyl-2-oxazoline).

Preferably, the backbone of said graft macromolecule comprises a first repeating unit which is hydrophilic, and the hydrophobic side chains are bound to the first repeating units. More preferably, the first repeating unit contains a carboxylic group, and the hydrophobic side chains are bioresorable.

Preferably, the backbone of said graft macromolecule is polyacrylate, poly(acrylic acid), poly(methacrylic acid), poly(butenedioic acid), polyhistidine, or poly(vinyl imidazole). More preferably, the backbone of said graft macromolecule is poly(methacrylic acid).

Preferably, the hydrophobic side chains comprise poly(lactide), poly(lactic acid), or polycaprolactone. More preferably, the hydrophobic side chains comprise poly(lactide).

Preferably, the backbone of said graft macromolecule further comprises a second repeating unit which is different from the first repeating unit, and the second repeating unit will cause the core collapse in responsive to a temperature change. More preferably, the second repeating unit of the backbone of said graft macromolecule is derived from a monomer of N-isopropyl acrylamide. Most preferably, the backbone of said graft macromolecule is a copolymer of N-isopropyl acrylamide and methacrylic acid.

Preferably, the polymeric micelle has a diameter of 50-200 nm.

Preferably, said diblock copolymer has a terminal functionality connected to an end of the hydrophilic polymeric segment, and said terminal functionality is a ligand able to be bound to a receptor on a surface of a tumor cell. More preferably, the ligand is a galactose residue.

Preferably, said diblock copolymer has a terminal functionality connected to an end of the hydrophilic polymeric segment, and said terminal functionality is a fluorescence group. More preferably, said fluorescence group is a fluorescein isothiocyanate.

Preferably, said diblock copolymer has a terminal functionality connected to an end of the hydrophilic polymeric segment, and said terminal functionality is one of the terminal functionalities is a dye. More preferably, said dye is a near infrared dye.

Preferably, said structure comprises a plurality of different block copolymers, and each block copolymer comprising a hydrophobic polymeric segment and a hydrophilic polymeric segment. More preferably, each of said plurality of different block copolymers is a diblock copolymer comprising a hydrophobic polymeric segment and a hydrophilic polymeric segment.

Preferably, the hydrophobic polymeric segments of said plurality of different block copolymers have a same repeating unit.

Preferably, the hydrophilic polymeric segments of said plurality of different block copolymers have a same repeating unit.

Preferably, the hydrophilic polymeric segments of said plurality of different block copolymers have different repeating units.

Preferably, said plurality of different block copolymers have different terminal functionalities connected to ends of the hydrophilic polymeric segments. More preferably, one of the terminal functionalities is a ligand able to be bound to a receptor of on a surface of a tumor cell. Most preferably, the ligand is a galactose residue. Alternatively, one of the terminal functionalities is a fluorescence group, for example a fluorescein isothiocyanate. Selectively, one of the terminal functionalities is a dye, for example a near infrared dye.

### BRIEF DESCRIPTION OF THE DRAWINGS

FIG. 1A is a plot showing average diameters and polydispersity indexes of the mixed micelles G1B1 prepared in Example 1 of the present invention as a function of the molar ratio of the diblock copolymer Block I at a fixed concentration of the graft copolymer Graft I (10.0 mg/L).

FIG. 1B is a plot showing average diameters and polydispersity indexes of the mixed micelles G1B1B2 prepared in Example 1 of the present invention as a function of the molar ratio of the diblock copolymer Block II at a fixed concentration of the graft copolymer Graft I (10.0 mg/L) and the diblock copolymer Block I (5.625 mg/L).

FIG. 2 is a transmission electron microscopy (TEM) image of the mixed micelle G1B1B2 prepared in Example 1 of the present invention (The scale bar is 500 nm).

FIG. 3 is a plot of the ratio of intensities (I₁/I₃) of the vibrational bands in the pyrene fluorescence spectrum of the mixed micelle G1B1B2 prepared in Example 1 of the present invention as a function of temperature at pH 4.0.

FIG. 4A is a TEM Image of the mixed micelles G1B1B2 prepared in Example 1 of the present invention before structural change (Scale bar=200 nm).

FIG. 4B is a TEM Image of the mixed micelles G1B1B2 prepared in Example 1 of the present invention before structural change (Scale bar=200 nm).

FIG. 5 shows the amounts of Dox released from the Dox-loaded mixed micelles prepared in Example 2 of the present invention under acidic and neutral conditions at 25 and 37° C.

FIG. 6 shows growth inhibition of HeLa cells treated with various concentrations of Dox-loaded mixed micelles prepared in Example 2 of the present invention, free Dox, and Dox-loaded Graft I micelles.

FIG. 7 shows a TEM image of the Dox-loaded multifunctional micelles prepared in Example 5 of the present invention.

FIG. 8 shows the amounts of Dox released from the Dox-loaded multifunctional micelles prepared in Example 5 of the present invention under acidic (pH 5.0) and neutral (pH 7.4) conditions at 37° C. Mean±sd (n=3).

FIG. 9 is a plot showing growth inhibition of HepG2 cells treated with the Dox-loaded multifunctional micelles prepared in Example 5 and Dox-loaded mixed micelles prepared in Example 2 of the present invention after 24 h and 48 h incubation in a positive control and a negative control.

FIG. 10 is a plot showing growth inhibition of HepG2 cells treated with the Dox-loaded multifunctional micelles prepared in Example 5 and Dox-loaded mixed micelles prepared in Example 2 of the present invention after 24 h and 48 h incubation in a positive control and a negative control with the inhibition assay (in the presence of 150 mM galactose).

### DETAILED DESCRIPTION OF THE INVENTION

In this invention, a multi-component micelle was prepared from graft and diblock copolymers; the differences between the CMCs of the copolymers are used to control the particle size. Additionally, the mixed micelle in this structure can be extended for many applications by manipulating and carefully designing each component. One such application is as an anticancer drug carrier. Intracellular drug delivery is one of the important routes for being used in cancer therapy. This pathway enhances the cytotoxicity of drugs toward targeted cells and minimizes the side effects on normal tissue. The mechanisms for inducing the release of drugs from carriers after the uptake by cells involve lysosomal enzymes and a change in intracellular pH to deform the carriers. Many materials have been investigated and synthesized to achieve this pathway before the present invention. However, some of them, possessing hydrophobic groups or highly electronic charges, may be recognized by mononuclear phagocyte systems (MPS), cannot accumulate easily in tumor regions, and so are not suitable for use even in intravenous injection, far less intracellular drug delivery. Therefore, the hydrophilic segment extended on the surface of particle is necessary.

In one of the preferred embodiments of the present invention, a novel mixed micelle with a multi-functional core and shell was successfully prepared from an environmentally-sensitive graft copolymer, poly(N-isopropyl acrylamide-co-methacryl acid)-g-poly(D,L-lactide) (P(NIPAAm-co-MAAc)-g-PLA) and two diblock copolymers, methoxy poly(ethylene glycol)-b-poly(D,L-lactide) (mPEG-PLA) and poly (2-ethyl-2-oxazoline)-b-poly(D, L-lactide) (PEOz-PLA). This nano-structure completely screens highly negative charges of the graft copolymer and exhibits multi-functions because it has a specialized core-shell structure. An example of this micelle structure in intracellular drug delivery demonstrated a strong relationship between drug release and the functionality of the mixed micelle. Additionally, the efficiency of screening feature also displayed in the cytotoxicities; mixed micelles exhibited higher drug activity and lower material cytotoxicity than micelles from P(NIPAAm-co-MAAc)-g-PLA ([NIPAAm]/[MAAc]/[PLA]=84:5.9:2.5 mol/mol). This embodiment not only presents a new micelle structure generated using a graft-diblock copolymer system, but also elucidates concepts on which the preparation of a multi-functional micelle from a graft copolymer with a single or many diblock copolymers can be based for applications in drug delivery.

In another preferred embodiments of the present invention, multifunctional micelles for cancer cell targeting, distribution imaging, and anticancer drug delivery were prepared from an environmentally-sensitive graft copolymer, P(NIPAAm-co-MAAc)-g-PLA, a diblock copolymer, mPEG-PLA and two functionalized diblock copolymers, galactosamine-PEG-PLA (Gal-PEG-PLA) and fluorescein isothiocyanate-PEG-PLA (FITC-PEG-PLA). Multifunctional micelles target specific tumors by an asialoglycoprotein (HepG2 cells)-Gal (multifunctional micelle) receptor-mediated tumor targeting mechanism. The intracellular pH changes then induce structural deformation of the P(NIPAAm-co-MAAc)-g-PLA graft copolymer inner core of multifunctional micelles and thereby increases HepG2 cell cytotoxicity by releasing doxorubicin (Dox). Confocal laser scanning microscopy (CLSM) reveals a clear distribution of multifunctional micelles. With careful design and sophisticated manipulation, polymeric micelles synthesized in the present invention can be widely used in cancer diagnosis, cancer targeting, and cancer therapy simultaneously.

The present invention will be better understood through the following examples which are merely for illustrative and not for the limitation of the scope of the present invention.

### EXAMPLE 1

### Materials.

D,L-Lactide and methacrylic acid (MAAc) were purchased from Lancaster. Methyl p-toluenesulfonate (MeOTs), stannous octoate, 2-hydroxyethyl methacrylate (HEMA), pyrene and 2,2′-azobisisobutyronitrile (AIBN) were purchased from Aldrich. N-Isopropyl acrylamide (NIPAAm) and 2-ethyl-2-oxazoline were purchased from TCI. MPEG (weight-average molecular weight, Mw=5000 Da) was purchased from Sigma. D,L-Lactide was further purified by recrystallization from tetrahydrofuran (THF) twice before used. NIPAAm and AIBN were purified by recrystallization from hexane and acetone, respectively. MAAc and HEMA were purified by distillation under vacuum. 2-Ethyl-2-oxazoline and MeOTs were treated with CaH₂ overnight and purified by distillation under vacuum. Other reagents were commercially available and were used as received.

### Preparation of Graft Copolymer P(NIPAAm-co-MAAc)-g-PLA (Graft I, G1)

First, PLA with an end-capping, methacrylated group (PLA-EMA) was synthesized by ring-opening polymerization. D,L-Lactide (4 g), HEMA (0.26 g) and toluene (5 mL) were added to a two-necked round-bottle flask with magnetic stirring. The flask was immersed in an oil bath and stirred at 130° C. under nitrogen. Stannous octoate (1 wt %) was then added to start the polymerization, which was continued for 16 h at 100° C. After polymerization, the product was terminated by adding 0.1 N methanolic KOH and then precipitated from diethyl ether twice. PLA-EMA with one end capped by a methacrylated group was obtained (Mn=2000). P(NIPAAm-co-MAAc)-g-PLA graft copolymer was synthesized by traditional free-radical copolymerization. PLA-EMA (0.35 g), NIPAAm (1.15 g), MAAc (0.16 g) and AIBN (0.023 g) were placed in a two-necked round-bottle flask with a magnetic stirring bar, and the mixture was dissolved in acetone (15 mL). The reaction was performed at 70° C. for 24 h under nitrogen. After polymerization, the product was purified twice by precipitation from diethyl ether and twice by precipitation from distilled water, to yield the final graft copolymer (P(NIPAAm-co-MAAc)-g-PLA ([NIPAAm]:[MAAc]:[PLA]=84:5.9:2.5 mol/mol (Graft I, G1).

### Synthesis of Diblock Copolymer MPEG-PLA (Block I, B1)

MPEG-PLA diblock copolymer was synthesized by ring-opening polymerization. D,L-Lactide (1 g), mPEG (Mw=5000 Da) (10 g) and toluene (4 mL) were added to a two-necked round-bottle flask with a magnetic stirring bar. The mixture was heated in an oil bath and stirred at 130° C. under nitrogen. Stannous octoate (1 wt %) was then added to start the polymerization, which was continued for 16 h at 130° C. After polymerization, the product was terminated by adding 0.1 N methanolic KOH and recrystallizing from dichloromethane and diethyl ether cosolvent at −20° C. m PEG-PLA ([EG]:[LA]=113:7 mol/mol) was thus obtained (Block I, B1).

### Synthesis of Diblock Copolymer PEOz-PLA (Block II, B2)

PEOz-PLA was prepared by the modification of procedures in the literature [G. H. Hsiue, C. Ch. Wang, C. L. Lo, C. H. Wang, J. P. Li, J. L. Yang, Int. J. Pharm. 2006, 317, 69], as follows. First, 2-ethyl-2-oxazoline (10 mL), the initiator methyl p-toluenesulfonate (0.232 mg) and acetonitrile (30 mL) were added to a two-necked round-bottle flask with a magnetic stirring bar. The flask was moved to an oil bath and the mixture was stirred at 100° C. under nitrogen for 30 h. After cooling to room temperature, the reaction was terminated by adding 0.1 N methanolic KOH and precipitating twice from diethyl ether twice to yield PEOz-OH. Then, PEOz-OH (2 g) and D,L-lactide (0.426 g) were polymerized using stannous octoate (1 wt. %) for 16 h at 130° C. under nitrogen. After polymerization, the product was terminated by adding 0.1 N methanolic KOH and precipitating twice from diethyl ether to yield PEOz-PLA ([EOz]:[LA]=52.5:9.7 mol/mol) (Block II, B2).

The chemical structure and polydispersity index of each copolymer prepared above were verified by ¹H-NMR (AMX-500, Bruker) and GPC using dimethylformamide (DMF) as an elution solvent. The Mn of Graft was calculated by ¹H-NMR (AMX-500, Bruker) using mPEG (Mn 2000) as a standard. Additionally, the critical micelle concentration (CMC) of each was identified using a fluorescence spectrometer with pyrene as a hydrophobic probe. The copolymer concentration varied from 0.0001 to 10 mg/mL. Fluorescence spectra were obtained using a fluorescence spectrophotometer (F-2500, Hitachi). The excitation wavelength for the emission spectra was 339 nm and excitation spectra were recorded at 390 nm. Table 1 summarizes those results.

TABLE 1

| | Mn [Da] | Mn [Da] | | |
|---|---|---|---|---|
| | hydrophilic | hydrophobic | Polydispersity | CMC |
| | segment | segment | index | [g/mL] |
| Graft I | 9970 | 6150 | 1.24 | 1.27 × 10⁻⁶ |
| Block I | 5000 | 500 | 1.05 | 8.39 × 10⁻⁵ |
| Block II | 5200 | 700 | 1.16 | 1.70 × 10⁻⁶ |

### Preparation of Micelles from P(NIPAAm-co-MAAc)-g-PLA, mPEG-PLA and PEOz-PLA

Various compositional ratios of Graft I and Block I, with or without Block II, were dissolved together in DMSO to prepare a polymer solution. The polymer solution was then dialyzed against distilled water for 48 h at 20° C. using a cellulose membrane bag (with a molecular weight cut-off of 6000-8000, obtained from SpectrumLabs, Inc.). The distilled water was replaced every 3 h. After dialysis, micelle or mixed micelle solutions were collected and frozen using a freeze dryer system (Heto-Holten A/S, Denmark) to yield dried products.

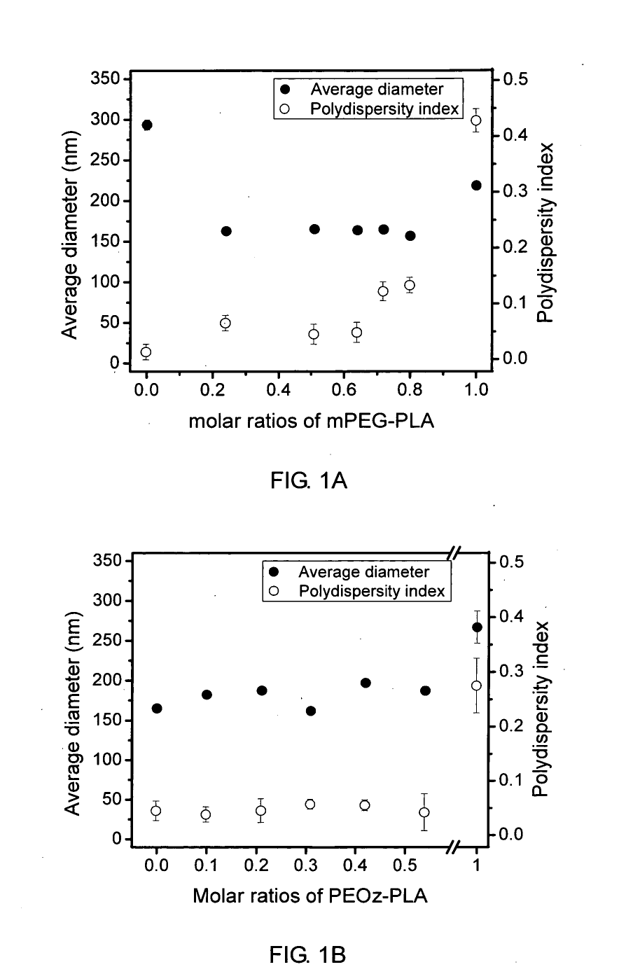

FIG. 1A is a plot showing average diameters and polydispersity indexes of mixed micelles G1B1 as a function of the molar ratio of Block I at a fixed concentration of Graft I (10.0 mg/L). FIG. 1B is a plot showing average diameters and polydispersity indexes of mixed micelles G1B1B2 as a function of the molar ratio of Block II at a fixed concentration of Graft I (10.0 mg/L) and Block I (5.625 mg/L).

Table 2 lists the concentrations of Graft I, Block I and Block II used in preparing two of the micelles shown in FIGS. 1A and 1B.

TABLE 2

| | G1 (mg/L) | B1 (mg/L) | B2 (mg/L) |
|---|---|---|---|
| G1:B1:B2 = 33.9:55.7:10.4 mol/mol | 10.0 | 5.625 | 1.125 |
| G1:B1 = 50:50 mol/mol | 10.0 | 3.15 | — |

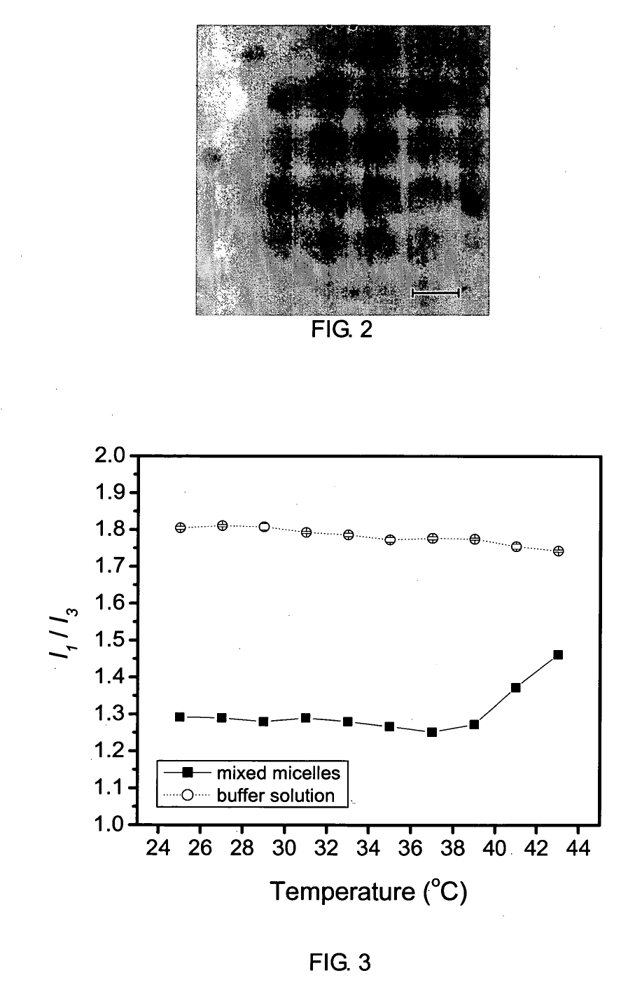

Three copolymers of the mixed micelle G1B1B2 [G1:B1:B2=33.9:55.7:10.4 mol/mol] exhibited self-assembly, packing and association with hydrophobic PLA to form mixed micelles yielding a uniform particle size (182.3±1.5 nm) and a narrow distribution (polydispersity index, PDI=0.038±0.014), as determined by dynamic light scattering (DLS) from the sample in phosphate buffer saline (PBS) at a concentration of 0.1 mg/mL. The zeta-potential of the mixed micelle was measured by Doppler microelectrophoresis (Zetasizer 3000HS, Malvern) in PBS at a concentration of 0.1 mg/mL, to identify the effect of diblock copolymers on hiding efficiency. The micelle that was composed of Graft I was used as a comparative sample; the corresponding zeta-potential was measured to be −15.5±0.9 mV. The highly negative charge caused by the slight ionization of carboxyl acid groups of MAAc was screened by diblock copolymers in the mixed micelle. The zeta-potential of the mixed micelle was measured to be −7.8±1.3 mV, because the hydrophilic segments MPEG and PEOz were extended on the surface of the mixed micelles, hiding the carboxyl acid groups of MAAc. The most direct evidence of the mixed micelle structure is obtained by transmission electron microscopy (TEM; Hitachi H-600 microscope, accelerating voltage=100 kV), as shown in FIG. 2. TEM is commonly employed to identify the core-shell structure of mixed micelles by the staining of methacrylic acid groups by uranyl acetate (2 wt %). The TEM image of the mixed micelle G1B1B2 suggests that the dark region of the Graft I copolymer is the inner core, and that the hydrophilic segments of mPEG and PEOz extend outside the Graft I core. Additionally, a Bioscope AFM was used to observe the shape of the mixed micelle, and the results indicate that the mixed micelle G1B1B2 are of uniform size and are spherical. The size of the particles was similar to that determined by DLS.

Mixed micelles were dialyzed from Graft I with Block I (G1B1) and from Graft I and Block I with Block II (G1B1B2), to compare the effects of the compositions of the diblock copolymers on the preparation of mixed micelles. The three copolymers exhibited various CMCs: the CMC of Block I differed greatly from that of the Block II and Graft I (Table 1). When a fixed concentration of Graft I was treated with various molar ratios of Block I (CMC_{Graft I}<<CMC_{Block I}), the average diameters of the G1B1 mixed micelles were smaller than those from single Graft I or single Block I, and remained constant at around 160 nm, as determined by DLS, as shown in FIG. 1A. The size distributions of the mixed micelles G1B1 were narrow. However, when Block II was added to a fixed concentration of G1B1, whereas the CMC of Block II was nearly that of Graft I but smaller than that of Block I, the average diameters of the G1B1B2 mixed micelles were independent of Block II, as shown in FIG. 1B; the sizes differed greatly from those of the micelles from one copolymer or mixed micelles from Graft I with Block II (330.2±0.9 nm; PDI=0.072±0.011). However, they were close to the size obtained from the G1B1 mixed micelles. When the molar ratio of Block II exceeded 0.54, no G1B1B2 mixed micelle was obtained—the copolymers aggregated and precipitated as dialysis began. These results indicate that the copolymer with the highest CMC determined the particle size. That is, the high CMC of the diblock copolymer in the graft-diblock copolymer system, such as Block I in the presented system, helps to regularize and control the formation of the mixed micelles, controlling and reducing the size of the particles. The relative CMCs are therefore important considerations in preparing mixed micelles of small size and low PDI from graft and diblock copolymers.

Poly(N-isopropylacrylamide) (PNIPAAm) is well known to be a water-soluble and hydrophilic polymer, that exhibits an extended chain conformation below the lower critical solution temperature (LCST) when it is in aqueous solution. PNIPAAm can also undergo a phase transition to an insoluble and hydrophobic aggregate above its LCST. Randomly copolymerizing a small proportion of the MAAc in PNIPAAm copolymers raises the LCST above 37° C. (i.e., body temperature) and causes the polymer to be sensitive to pH. P(NIPAAm-co-MAAc)s exhibits an extended chain in neutral surroundings. This is because the ionized MAAcs increase the hydrophilicity of P(NIPAAm-co-MAAc)s. In acidic surrounding, the copolymer aggregates and precipitates, owing to the fact that the de-ionized MAAcs decreases the hydrophilicity of P(NIPAAm-co-MAAc)s and reduces its LCST to 32° C. The pH-sensitive properties of MAAc and thermal-sensitive properties of PNIPAAm are correlated. Our previous study demonstrated that Graft I micelles exhibited a structural change because of aggregation and the collapse of the P(NIPAAm-co-MAAc) outer shells in response to the change of the temperature at low pH [C. L. Lo, K. M. Lin, G. H. Hsiue, J. Controlled Release 2005, 104, 477]. FIG. 3 presents the structural change of the mixed micelles G1B1B2 [G1:B1:B2=33.9:55.7:10.4 mol/mol] as a function of temperature at pH 4.0. The structural change in the mixed micelles was determined by fluorescence spectrometry using pyrene as a hydrophobic probe. The ratio I₁/I₃, of the intensity of the first vibrational band to that of the third can then be used as an index of environmental polarity [K. Kalyanasundaram, J. K. Thomas, J. Am. Chem. Soc. 1997, 99, 2039]. A higher ratio corresponds to the pyrene probe being in more polar surroundings. The pyrene buffer solution was used as a reference for comparison with the change of I₁/I₃ before and after the structural deformation of the mixed micelles. The I₁/I₃ ratio of the pyrene buffer solution decreased gradually from 1.81 to 1.74 as the temperature increased because of thermal decay. For mixed micelles at pH 4.0, the I₁/I₃ of the pyrene spectra began to decrease at approximately 31° C., perhaps because the pyrene molecules were partitioned in the hydrogen-bonding region between MPEG and MAAc. As the temperature increased above 37° C., I₁/I₃ rapidly increased from 1.25 to 1.46, indicating that the surroundings of pyrene changed from the mixed micelle inner core to the buffer solution, because of structural deformation.

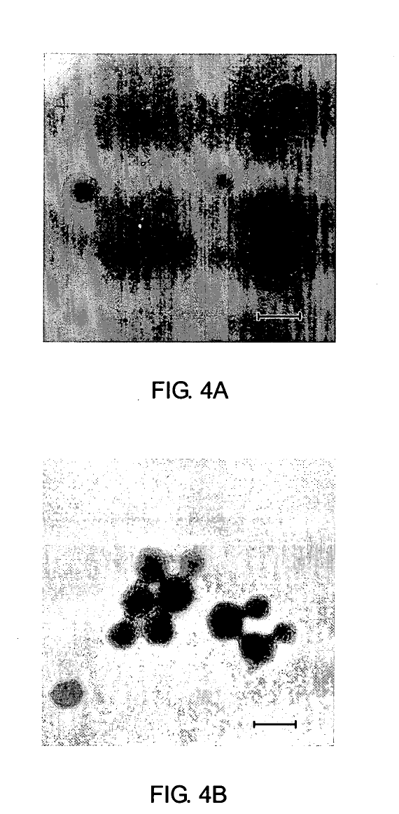

The inventors of the present invention also analyzed mixed micelles G1B1B2 [G1:B1:B2=33.9:55.7:10.4 mol/mol] before and after structural changes by using time-of-flight secondary ion mass spectrometry (TOF-SIMS). The chemical compositions of the surface layers of mixed micelles before and after the induced structural changes were determined from positive and negative TOF-SIMS spectra. The results from TOF-SIMS spectra indicate that the Graft I in the mixed micelles was exposed and closed the surface layer after the induced structural change. The mixed micelles after structural change were treated with uranyl acetate and monitored by TEM to yield further evidence of this finding. As shown in FIGS. 4A and 4B, the TEM images demonstrate that the core-shell structure of the mixed micelles G1B1B2 [G1:B1:B2=33.9:55.7:10.4 mol/mol] was destroyed. The dark region of Graft I was in both the inner core and the outer shell for mixed micelles after structural change.

### EXAMPLE 2

In this example, a mixed micelle structure, composed of MPEG-PLA diblock copolymer and P(NIPAAm-co-MAAc)-g-PLA graft copolymer, was used to encapsulate a hydrophobic anticancer drug, free base doxorubicin (Dox), whose structure enables the encapsulated drug to remain in the core during circulation in the blood.

Doxorubicin (Dox)-loaded mixed micelle was also prepared by dialysis. The preparation procedures were similar to those of the mixed micelles prepared in Example 1. 20 mg of Dox-HCl was dissolved in 8 ml DMF and 2 ml DMSO. 2 mg of mPLA-b-PEG (Block I) and 20 mg of P(NIPAAm-co-MAAc)-g-PLA (Graft I) were dissolved in 8 ml DMF and 2 ml DMSO. The Dox-HCl solution was mixed with 0.3 ml of triethylamine to remove hydrochloride. Then, the free base Dox solution was added to the polymer solution and stirred at room temperature for 2 h. The mixed solution was dialyzed against water at 20° C. for 72 h. The distilled water was replaced every 3 h. After dialysis, the solution of micelles was collected and frozen using a freeze-drying system to yield dried micelles. Weighted amounts of the mixed micelles were dissolved in DMSO at room temperature for 12 h; they then underwent ultrafiltration (ultrafiltration membrane MWCO 1000, Millipore) and samples were removed and analysis to determine Dox content using a UV/Vis spectrometer at 485 nm by reference to a calibration curve of Dox in DMSO. Accordingly, the Dox content in the mixed micelles was determined. The drug content of mixed micelles was calculated using the formula: drug content (% w/w)=(total mass of Dox in mixed micelles)/(total mass of Dox in mixed micelles+total mass of polymer in mixed micelles)×100.

The mixed micelles incorporated with Dox (Dox-loaded mixed micelles) were formed with a uniform particle size of about 165 nm as shown in an AFM image.

Drug Release Assay. The release of Dox-loaded mixed micelles in pH 5.0 and pH 7.4 buffer solutions at 37° C. and 25° C., respectively, was examined. Dox released from mixed micelles was isolated from the mixed-micelle buffer solution (50 mg/L) by ultrafiltration (ultrafiltration membrane MWCO 10000, Millipore). The isolated solution was measured using a UV/Vis spectrometer at 485 nm in a time-course procedure.

Cytotoxicity Evaluation. The cytotoxicity of each sample was determined by measuring the inhibition of cell growth using a tetrazolium dye (MTT) assay. Dox-loaded mixed micelles and Dox-loaded Graft I micelles were washed twice with PBS to remove untrapped Dox before use. HeLa cells (5×10³ cell/mL) harvested in a logarithmic growth phase were seeded on 96 wells in Dulbecco's modified Eagle's medium (DMEM) with 10% fetal bovine serum (FBS) in a humidified atmosphere of 5% CO₂ at 37° C. After the HeLa cells had been incubated in a logarithmic growth phase, samples with various concentrations of Dox were added for 48 h of co-culturing. At the end of the experiment, the MTT assay was conducted and the percentage of cell viability was calculated. Additionally, material cytotoxicities were measured using HeLa cells (5×10³ cell/mL). The experimental process was identical to that described above.

Internalization Evaluation. Accumulated Dox in HepG2 cells was localized using a Carl Zeiss LSM5 PASCAL confocal laser scanning microscope (CLSM). The HeLa cells were seeded on coverslides for 24 h and then treated with free Dox or Dox-loaded mixed micelles. Dox-loaded mixed micelles were washed with PBS twice to remove untrapped Dox before use. The concentration of Dox was ca. 10 μg/mL. After an interval, the cells were washed twice with PBS; then, the LysoTracker was added in a culture medium without FBS. After 30 min of incubation, the cells were washed with PBS and mounted on a slide with 4 wt % paraformaldehyde for CLSM observation. Fluorescence observation was carried out with a confocal microscope at 488 nm for excitation and an LP (long-pass) filter of 590 nm for Dox detection. Besides, LysoTracker observation was also carried out with a confocal microscope at 504 nm for excitation and an LP filter of 511 nm for detection.

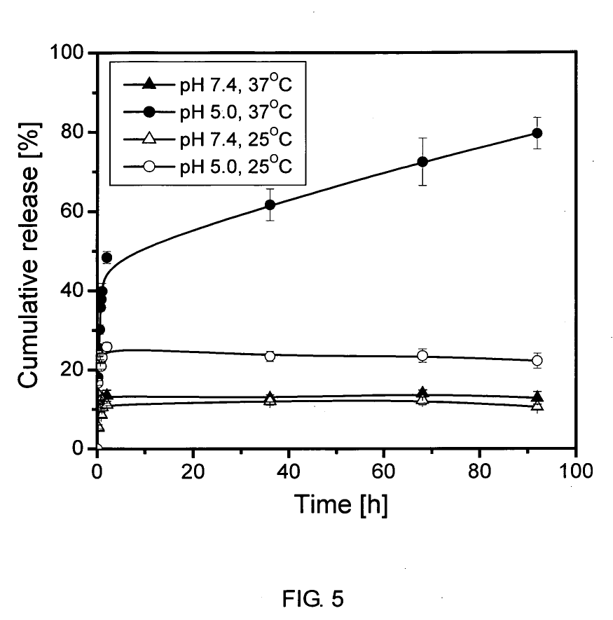

UV/Vis spectrophotometry demonstrated that the drug content of the mixed micelle incorporating Dox was around 19%. The Dox released from mixed micelles was isolated from the micellar buffer solution using the ultrafiltration membrane. FIG. 5 shows the amount of Dox released from mixed micelles at different pH levels. In neutral surroundings, mixed micelles with Dox exhibited an initial burst that released about 15% of Dox at 37 or 25° C. In acidic surroundings at 25° C., mixed micelles maintained their complete cores and the release of Dox approached 25%, perhaps because that the hydrogen-bonding occurred between MAAc and MPEG, compressing the core and releasing Dox. In contrast, almost 50% of the Dox was released from mixed micelles in the initial 2 h at pH 5.0 and 37° C., because the P(NIPAAm-co-MAAc) collapsed and aggregated in acidic surroundings, deforming the inner core, causing Dox to be released from the mixed micelles. These results strongly demonstrate that structural deformation controls drug release.

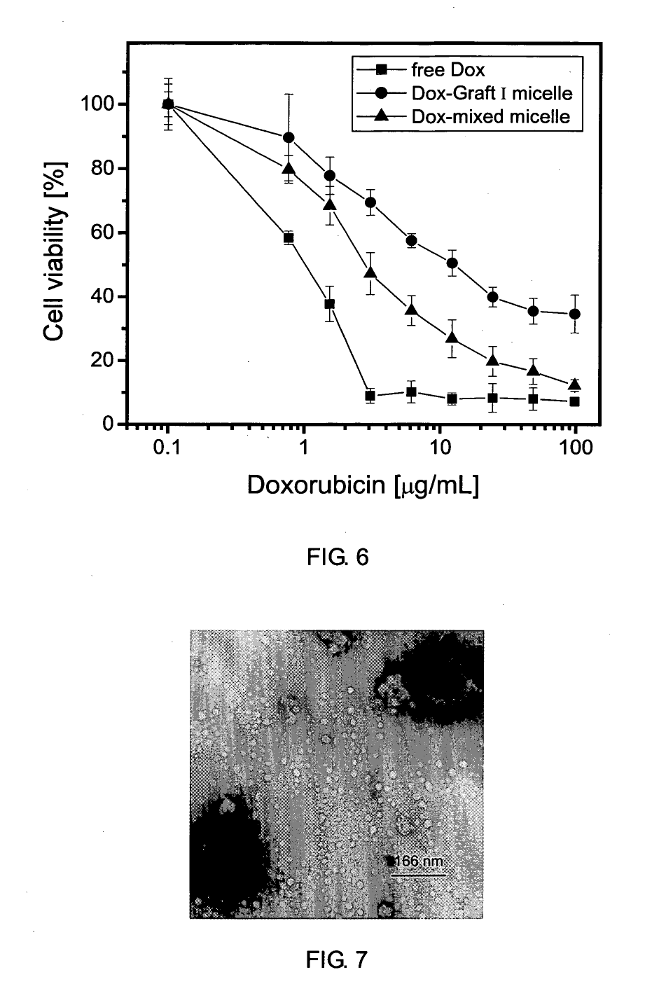

HeLa cells (5×10³ cell/ml) were used to study the cytotoxicity of free Dox and Dox-loaded mixed micelle by measuring the inhibition of cell growth using a tetrazolium dye (MTT) assay. Micelle that incorporated Dox from Graft I copolymer (Dox-loaded Graft I micelle) with a particle size of 176.2 nm and a drug content of about 17% was used for comparison. As shown in FIG. 6, free Dox exhibited more potent activity than the Dox-loaded mixed micelles and Dox-loaded Graft I micelles, because the cumulative release of Dox from mixed micelles or Graft I micelles after 48 h incubation approached 65%, so the total amount of active Dox exceeded that of Dox-loaded mixed micelles or Dox-loaded Graft I micelles. Moreover, the IC₅₀ (half maximal inhibitory concentration) of Dox-loaded Graft I micelles was found to be around 6 μg/mL, higher than that of Dox-loaded mixed micelles (about 3 μg/mL), because the highly negative charge of MAAc surrounded the surface of the Graft I micelle, resulting in a repulsive force between the micelles and the HeLa cell, reducing uptake and accumulation in HeLa cells. The empty mixed micelles and Graft I micelles were also treated with HeLa cells to evaluate the IC₅₀ of each one: these were found to be approximately 2.5 mg/mL and 1.5 mg/mL, respectively. The difference between the cytotoxicity of Graft I micelles and that of mixed micelles follows from the fact that the compositional proportion of Graft I in mixed micelles was slightly less than that in Graft I micelles at the same treatment concentration. These results demonstrates that the empty Graft I micelles are toxic. That is, the applications of the Graft I micelles in cancer therapy and intracellular drug delivery are limited. Introducing a steric and stabilized diblock copolymer into the micelle structure, in which the negative charge of MAAc is hidden, not only increases the cell uptake and decreases cytotoxicity, but also overcomes the limitations on polyions used in intravenous injection.

Confocal laser scanning microscopy (CLSM) was used to observe the intracellular drug release of mixed micelles. Confocal images were taken to observe the time-dependent fluorescence intensity of LysoTracker and Dox after mixed micelles were incubated with HepG2 (human hepatocellular carcinoma) cells. The red fluorescence from Dox and the green fluorescence from LysoTracker were detected in the intracellular compartment. A LysoTracker molecule was an indicator while located in the acidic compartment. After both one hour and eight hours incubation of HepG2 with Dox, the fluorescence from free Dox was concentrated in the nucleus. Fluorescence from LysoTracker occurred in both the nucleus and cytoplasm because these are acidified by doxorubicin hydrochloride. After Dox-loaded mixed micelles had been exposed for 1 h, a small amount of Dox was released from the mixed micelles and observed in the cytoplasm, where the LysoTracker molecules were also located, indicating that the mixed micelles were taken up from the extracellular fluid into the cells by endocytosis, and the pH of the endosomal compartments were then changed, inducing the release of Dox. Eight hours later, Dox was released from mixed micelles, associated with a strong signal. Dox was localized not only in the cytoplasm but also accumulated in the nucleus. Similar results were obtained when Chinese hamster ovary cells (CHO-K1) were treated with Dox-loaded mixed micelles.

The Dox-loaded mixed micelles prepared in this example can rapidly be damaged to release Dox when the intracellular pH changes; it also has a hydrophilic outer shell that screens highly negative charges and increases its solubility.

### EXAMPLE 3

Similar to the procedures in Example 1, Block III (mPEG₅₀₀₀-PLA₁₀₈₈, PDI=1.15, CMC=16 mg/L) and Block IV (mPEG₅₀₀₀-PLA₁₇₅₀, PDI=1.20, CMC=5.4 mg/L) copolymers were synthesized by ring-opening polymerization from methoxy poly(ethylene glycol) (mPEG, Mn 5000) and D,L-lactide using stannous octoate as a catalyst. These diblock copolymers have the same chemical nature, but differ in composition ratio.

Two-component mixed micelles composed of a graft copolymer (Graft I prepared in Example 1) and a diblock copolymer (Block I, Block III or Block IV) were employed to investigate the influence of chain length and CMC of the diblock copolymers on the morphology and structure of mixed micelles. First, a graft copolymer and a diblock copolymer were dissolved together in dimethylsulfoxide (DMSO)/dimethylformamide (DMF) (4/1 v/v) cosolvent to prepare a polymer solution. The DMF/DMSO solvent mixture was used because it produces the smallest mixed micelles. Graft copolymer concentration was fixed at 10 mg/mL. The molar ratio of the graft copolymer to the diblock copolymer was 1:9. Mixed micelles were then prepared by dialysis by using the procedures described in Example 1. The core-shell structure and particle size of three mixed micelles from a graft copolymer and a diblock copolymer (Block I, Block III or Block IV) were observed by transmission electron microscopy (TEM). TEM observation produced three results. (1) For all mixed micelles, the dark region of the graft copolymer is the inner core, and hydrophilic segments of mPEG extended outside the core. (2) The radius of the core region decreased as the chain length of PLA of diblock copolymer increased (PLA₅₀₀>PLA₁₀₈₈>PLA₁₇₅₀). (3) Mixed micelle particle size increased as the chain length of PLA of diblock copolymer increased (PLA₁₇₅₀>PLA₁₀₈₈>PLA₅₀₀). A short PLA length produces smaller mixed micelles.

A test for the stability of micelles in the presence of serum or serum albumins was conducted. In this test, mixed micelles (25 mol % of graft copolymer (Graft I) and 75 mol % of mPEG₅₀₀₀-PLA₁₇₅₀ (Block IV)) was chosen. The stability of mixed micelles was determined by dynamic light scattering (Zetasizer 3000HS, Malvern). Mixed micelles in PBS (2 mg/mL) were mixed with an equal volume of 4 wt % bovine serum albumin (BSA) dissolved in PBS. The mixture was incubated at 37° C. and determined by dynamic light scattering (DLS) at time interval, defined as tᵢ. The CONTIN analytic method was used. The average diameter of micelles in PBS (1 mg/mL) before BSA treatment, to was also measured. The ratio of particle sizes was calculated as tᵢ/t₀. Results show that mixed micelles were stable after 72 h because the hydrophilic outer shell MPEG prevented albumin adsorption on mixed micelles. This is one indication that mixed micelles could prolong the circulation after intravenous injection.

### EXAMPLE 4

Two functional end-capped diblock copolymers galactosamine (Gal)-PEG₃₄₀₀-PLA₈₃₀ (Gal-PEG-PLA, [Gal]: [PEG]: [LA]=8.4.:7.6:84 mol/mol) and fluorescein isothiocyanate (FITC)—PEG₃₄₀₀-PLA₈₃₀ (FITC-PEG-PLA, [FITC]: [PEG]: [LA]=4:8:88 mol/mol) were synthesized by thiol-maleimide coupling reaction.

FITC-PEG-PLA Diblock Copolymer Synthesis. PLA-NH₂. N-Boc-L-alaninol was converted to the corresponding alkoxide (N-Boc-L-alaninol-OK) using potassium/naphthalene. D,L-lactide (2 g) was then polymerized at 100° C. for 12 h using N-Boc-L-alaninol-OK (0.35 g) as an initiator and toluene (2 mL) as the solvent to obtain PLA-NHBoc. The polymerization was terminated by adding acetic acid to the reaction mixture and PLA-NHBoc was precipitated from diethyl ether. The Boc group was removed from the PLA-NHBoc (2.1 g) by treating with a mixed solvent of formic acid (20 mL) and CHCl₃ (20 mL). After 9 h treatment at room temperature, the solution was poured into a large amount of diethyl ether to obtain the precipitate. The precipitate was vacuum dried at room temperature. The product (1.5 g) was then deprotonated in a mixed solvent of triethylamine (20 mL) and CHCl₃ (20 mL) at room temperature for 8 h. PLA-NH₂ was purified by a method similar to that for PLA-NHBoc. PLA-SH. Thiolated PLA was synthesized by covalent modification of the primary amino groups of PLA-NH₂ by adding sulhydryl moieties. For the synthesis, PLA-NH₂ (2 g) was dissolved in acetonitrile (10 mL) and then reacted with an excess of 2-iminothiolane hydrochloride (0.458 g) at room temperature for 15 h. The unreacted 2-iminothiolane was removed by repeated dialysis against 5 mM HCl solution followed by 1 mM HCl solution for 24 h each. The purified PLA-SH was vacuum dried. Maleimide-PEG-NH₂. Aliquots of N-Methoxycarbonylmaleimide (0.2 g) in dimethyl sulfoxide (DMSO) (10 mL) were added to an aqueous solution of polyoxyethylene bis(amine) (1 g) at room temperature. The mixture was allowed to react for 6 h. After the reaction, the resulting Maleimide-PEG-NH₂ was purified by recrystallization from a mixed solvent of dichloromethane and diethyl ether (1/1 vol/vol) at −20° C. PLA-PEG-NH₂. PLA-SH (0.8 g) was dissolved in 0.1 M Tris/acetonitrile (1/3 v/v) (aq, pH 6.5, adjusted by 0.5 M NaOH solution) (15 mL) and then added to Maleimide-PEG-NH₂ (2 g) Tris solution (10 mL). The reaction mixture was shaken and allowed to continue for 6 h at room temperature. After the reaction, the product was purified by dialysis against PBS and Milli-Q water using a cellulose membrane bag (molecular weight cut-off, 6000-8000; obtained from SpectrumLabs, Inc.) and then frozen in a freeze dryer system (Heto-Holten A/S, Denmark) to yield dried product. The dried product was dissolved in dichloromethane and purified by precipitation from diethyl ether to remove unreacted PLA-SH. NH₂—PEG-PLA was obtained under vacuum. FITC-PEG-PLA. NH₂—PEG-PLA (1 g) was dissolved in methanol (40 mL) and the fluorescein isothiocyanate (FITC) (0.15 g) was then added. The mixture was stirred for 24 h at room temperature. The reaction mixture was then dialyzed against 0.5 M NaCl solution followed by dialysis against Milli-Q water for 2 days to remove methanol solvent and unreacted small molecules. Dried FITC-PEG-PLA product was obtained by a freeze dryer system.

Gal-PEG-PLA Diblock Copolymer Synthesis. Gal-Maleimide. Aliquots of N-Methoxycarbonylmaleimide (0.68 g) in dimethyl sulfoxide (DMSO) (10 mL) were added to an aqueous solution of galactosamine hydrochloride (0.5 g) at room temperature. The mixture was allowed to react for 6 h. The resulting Gal-Maleimide was purified by precipitation from diethyl ether. PLA-PEG-SH. Thiolated PLA was synthesized from the PLA-PEG-NH₂ with the addition of sulhydryl moieties. For the synthesis, PLA-PEG-NH₂ (1 g) was dissolved in acetonitrile (15 mL) and reacted with an excess of 2-iminothiolane hydrochloride (0.1 g) at room temperature for 15 h. Unreacted 2-iminothiolane was removed by repeated dialysis against 5 mM HCl solution followed by 1 mM HCl solution for 24 h. The Milli-Q water was replaced every 3 h. The purified PLA-PEG-SH was vacuum dried. Gal-PEG-PLA. PLA-PEG-SH (1 g) was dissolved in methanol (15 mL) and Gal-Maleimide (0.1 g) was then added. The mixture was stirred for 24 h at room temperature. The reaction mixture was then dialyzed against 0.5 M NaCl solution followed by dialysis against Milli-Q water for 2 days to remove methanol solvent and unreacted small molecules. The dried Gal-PEG-PLA product was obtained by a freeze dryer system.

### EXAMPLE 5

Multifunctional micelle incorporated with Dox was prepared using the dialysis method. First, Dox was neutralized with a 1.2 molar excess of triethyl amine in DMSO/DMF (4/1 v/v). This mixture was stirred to dissolve the drug. Fifty mol % of Graft I, 20 mol % of Block IV, 15 mol % of Gal-PEG-PLA, and 15 mol % of FITC-PEG-PLA were then dissolved in the drug solution. The mixture was dialyzed against Milli-Q water for 72 h using a membrane with a molecular-weight cut-off of 6000-8000 at room temperature. The Milli-Q water was replaced every 3 h. Multifunctional micelles were obtained by a freeze-drying process. The DOX loading level was about 31 wt % in weight, which was determined by a UV/Vis spectrophotometer as multifunctional micelles dissolved in DMSO. FIG. 7 shows the TEM image of the Dox-loaded multifunctional micelles stained with uranyl acetate (2 wt %). These results demonstrate the integrity of the core-shell structure. The Dox-loaded multifunctional micelle particle size was approximately 160 nm. As mentioned, macromolecular transport across blood vessels has been shown to occur via open gaps (interendothelial junctions and transendothelial channels), caveolae, vesicular vacuolar organelles, and fenestrations. The pore cutoff size in most tumor studies was between 380 and 780 nm. Dox-loaded multifunctional micelles in this example are below 200 nm, and would extravasate through the passageways described. Particle size has also been found to significantly influence the organ distribution of PEG-coated nanoparticles. A diameter of less than 200 nm is required to avoid spleen filtering effects. Particle size might also determine the internalization mechanism. Large particles (up to 500 nm) enter the cell by receptor- and clathrin-independent endocytosis while smaller particles (<200 nm) could be internalized via coated pits through a non-specific clathrin-dependent process. Thus, the Dox-loaded multifunctional micelles in this example were approximately 160 nm in size, close to the typical required size under physiological conditions.

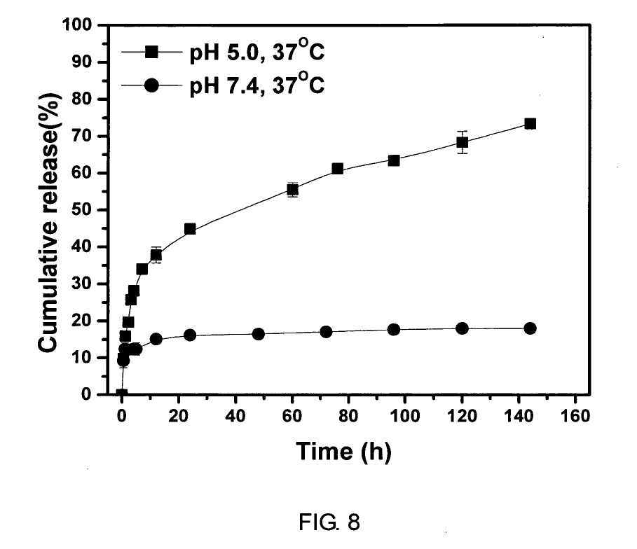

To evaluate the effects of stimulus-response behavior on controlled drug delivery, the in vitro drug release behaviors of the Dox-loaded multifunctional micelles were studied in two different buffered solutions (pH 7.4 and 5.0). FIG. 8 shows results. In neutral surroundings (pH 7.4), the Dox-loaded multifunctional micelles exhibited initial burst effects, losing about 15 wt % at 37° C. Release behavior remained constant after 140 h. In acidic surroundings (pH 5.0), release behavior was obviously divided into two periods. A rapid release in the first period was followed by a sustained and slow release over a prolonged time, up to a hundred hours for physically-encapsulated intelligence drug carriers. The initial rapid release (35 wt %) was observed in the initial 2 h and followed by a sustained release for 140 h until reaching a 70 wt % release profile. The drug release behavior results shown in FIG. 8 corroborate the claim that the Dox-loaded multifunctional micelles of the present invention are pH sensitive, and changing the pH will deform the core structure and released Dox.

Multifunctional micelles without Dox were also prepared for four components, including FITC-PEG-PLA, Gal-PEG-PLA, Block IV (mPEG₅₀₀₀-PLA₁₇₅₀) and Graft I copolymers by repeating the procedures of the preparation of the Dox-loaded multifunctional micelles except that Dox was not used. The graft copolymer (Graft I) in the multifunctional micelles could encapsulate anticancer drugs, and control drug release in response to pH or temperature changes. Block IV in micelles helped control the core-shell structure and obtain uniform micellar distribution. The fluorescence dye conjugated diblock copolymer FITC-PEG-PLA in micelles provided direct evidence of where micelles accumulated after cell uptake. On the other hand, the targeting moiety (Gal) conjugated diblock copolymer Gal-PEG-PLA could combine with the asialoglycoprotein of HepG2 cells in the active tumor targeting.

The Dox-loaded multifunctional micelles and free Dox were tested for in vitro cytotoxicity using a tetrazolium dye (MTT) method. The MTT-based cytotoxic activities of the Dox-loaded multifunctional micelles and free DOX were compared after 24 h and 72 h incubation with HeLa cells. The inhibition concentration (IC₅₀) of the Dox-loaded multifunctional micelles was 25 μg/1 mL at 24 h but decreased to 4 μg/mL at 72 h. The cytotoxicity of the Dox-loaded multifunctional micelles at 72 h was similar to free Dox (IC₅₀=1.2 μg/mL). On the other hand, the IC₅₀ Of empty multifunctional micelles was 792 μg/mL after 72 h of incubation. This indicates that the cytotoxicity of HeLa cells came from the Dox released by the Dox-loaded multifunctional micelles.

To evaluate the functionality of the Dox-loaded multifunctional micelles in biomarker applications, confocal laser scanning microscopy (CLSM) was used to observe the fluorescence images of the Dox-loaded multifunctional micelles and released Dox after HeLa cells uptake (for 6 h incubation). The triggering mechanism of most particulate carriers must occur in the endosome to release the drug in the cytoplasm. The CLSM fluorescence images show that HeLa cells showed green fluorescence in the cytoplasm, indicating that the multifunctional micelles were located there. Additionally, the released Dox, with a red fluorescence, was localized in both the cytoplasm and the nucleus. The clear pathway of where particulate carrier delivery was observed by the FITC-labeled micelles.

### EXAMPLE 6

To evaluate the functionality of multifunctional micelles in specific tumor targeting, the Dox-loaded multifunctional micelles prepared in Example 5 and Dox-loaded mixed micelles prepared in Example 2 were incubated with HepG2 (hepatocellular carcinoma) cells.

Tumor Targeting Evaluation. HepG2 cells (2×10⁴ cells/mL) were seeded in a 25-T flask of DMEM medium with 10% fetal bovine serum (FBS) in a humidified atmosphere of 5% CO₂ at 37° C. After the HepG2 cells had been incubated in a logarithmic growth phase, the Dox-loaded multifunctional micelles or the Dox-loaded mixed micelles were added for 2 h at 4° C. HepG2 cells were twice washed by PBS solution, and fresh medium was added for 24 h and 48 h incubation in a humidified atmosphere of 5% CO₂ at 37° C. At the end of the experiment, cell viability was calculated by trypan blue staining using a phase contrast microscopy (the positive control). The same process was repeated at 37° C. through the entire process as a negative control. The procedures were repeated except that galactose (150 mM) was also added to the system for performing an inhibition assay.

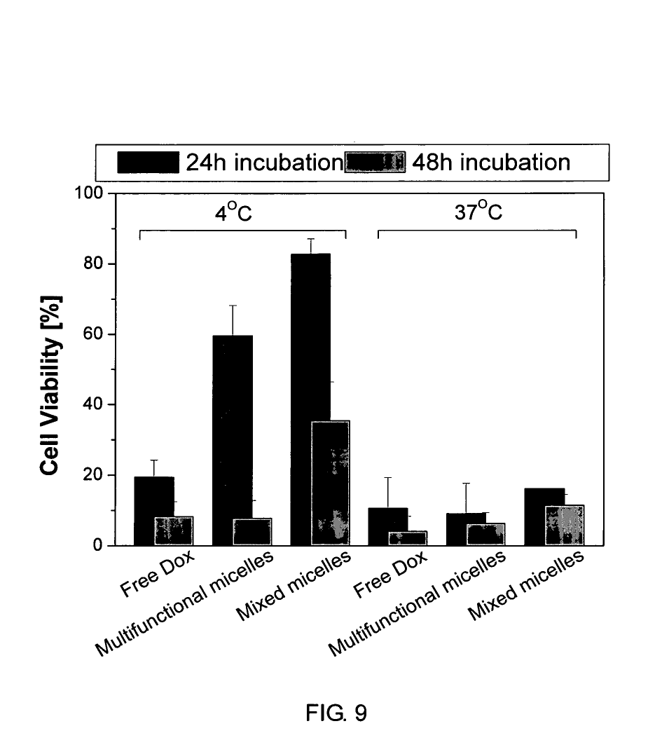

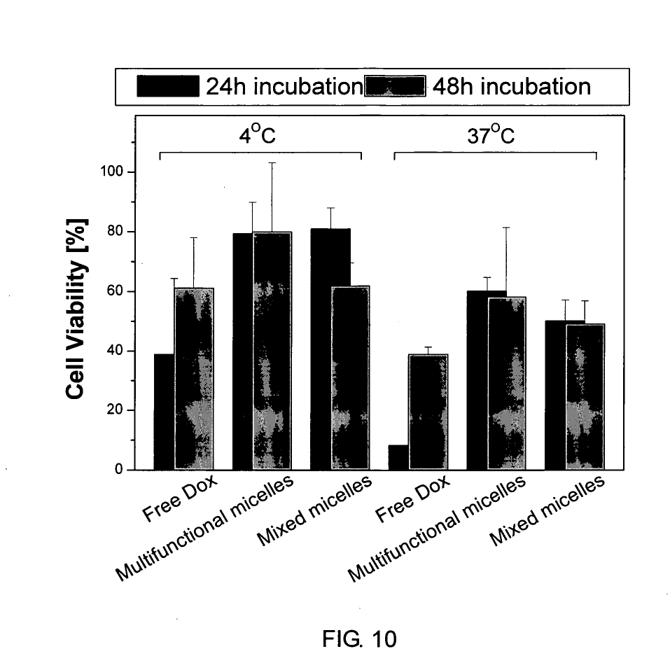

Hepatocytes have large numbers of asialoglycoprotein receptors on their surface that recognize galactose residues. Because of their specific ligand-receptor binding, the internalization of the Dox-loaded multifunctional micelles (containing the targeting moiety, Gal) into cancer cells can be performed by the receptor-mediated endocytosis process (active tumor targeting) and delivered to the lysosomes. The viability (percentage of surviving cells) of HepG2 cells after 24 h and 48 h incubation of the Dox-loaded multifunctional micelles was compared with the Dox-loaded mixed micelles. FIG. 9 shows the effects of specific tumor targeting and nonspecific tumor targeting of the micelles on receptor-mediated endocytosis. For the positive control, cells were incubated at 4° C. with the micelles to allow binding (but not internalization) to occur for 2 h. They were then replaced with fresh medium and warmed to 37° C. for various length of times. For the negative control, cells were incubated at 37° C. with micelles and underwent the same procedures as the positive control. The Dox-loaded multifunctional micelles had lower cell viabilities than the Dox-loaded mixed micelles without Gal in either the positive control or the negative control. This is because the Dox-loaded multifunctional micelles bound with asialoglycoprotein and then internalized into cancer cells to release Dox by intracellular pH changes. Additionally, at 37° C. the cell viability of all cells incubated with the Dox-loaded multifunctional micelles was lower than that of the cells incubated at 4° C., suggesting an endocytosis process and a large accumulation. The specific asialoglycoprotein-multifunctional micelle interactions were verified by an inhibition assay. The results of the inhibition assay is shown in FIG. 10. The incubation of cells with 150 mM galactose completely abolished micelle cell binding and indicated sugar specificity of the process involved.

It can be seen from Examples 5 and 6, multifunctional micelles encapsulating Dox were successfully prepared by dialysis, which can be used as cancer diagnosis agents and cancer drug delivery carriers. TEM images reveal that the Dox-loaded multifunctional micelles are spherical in shape and about 160 nm in size, which is suitable for intravenous injection and close to the typically required size under physiological conditions. Tumor targeting assay and CLSM measurements reveal that the Dox-loaded multifunctional micelles exhibit a high cytotoxicity by receptor-mediated endocytosis and show clear fluorescence imaging of their distribution. This shows a proof-of-concept: that is, producing an ideal micelle with a long circulation time, tumor recognition, and combined cancer diagnosis and controlled drug delivery for cancer therapy. It is apparent that multifunctional micelles combined with a near IR dye (e.g. Cy5.5) to replace FITC can be extended to animal models to evaluate the distribution in the body and cancer therapy.

### REFERENCES

[1] X. Gao, Y Cui, R. M. Levenson, L. W. K. Chung, S. Nie, Nat. Biotechnol. 2004, 22, 969.
[2] N. Kang, M. E. Perron, R. E. Prud'Homme, Y Zhang, G. Gaucher, J. C. Leroux, Nano lett. 2005, 5, 315.
[3] E. S. Lee, K. Na, Y H. Bae, Nano Lett. 2005, 5, 325.
[4] C. L. Lo, K. M. Lin, C. K. Huang, G. H. Hsiue, Adv. Funct. Mater (DOI: 10.1002/adfm.200500627)
[5] D. F. K. Shim, C. Marques, M. E. Cates, Macromolecules 1991, 24, 5309.
[6] C. Honda, K. Yamamoto, T. Nose, Polymer 1996, 37, 1975.
[7] A. L. Borovinskii, A. R. Khokhlov, Macromolecules 1998, 31, 7636.
[8] C. Konak, M. Helmstedt, Macromolecules 2003, 36, 4603.
[9] W. Mingvanisg, C. Chaibundit, C. Boot, PCCP 2002, 4, 778.
[10]T. Liu, V. N. Nace, B. Chu, Langmuir 1999, 15, 3109.

1. A polymeric micelle having a core-shell structure, wherein said structure comprises a graft macromolecule and a block copolymer, said graft macromolecule comprising a backbone and hydrophobic side chains bound to the backbone, said block polymer comprising a hydrophobic polymeric segment and a hydrophilic polymeric segment, wherein the hydrophobic side chains of said graft macromolecule are aggregated, and the hydrophobic polymeric segment of said block polymer is packed and associated to the aggregated hydrophobic side chains of the graft macromolecule with the hydrophilic polymeric segment of the block polymer extruding therefrom to form the core-shell structure.

2. The polymeric micelle according to claim 1, wherein the hydrophobic side chains and the hydrophobic polymeric segment comprise a same repeating unit.

3. The polymeric micelle according to claim 1, wherein the block copolymer is a diblock copolymer comprising the hydrophobic polymeric segment and the hydrophilic polymeric segment.

4. The polymeric micelle according to claim 3, wherein the hydrophobic polymeric segment has a number-average molecular weight of 500-2500, and the hydrophilic polymeric segment has a number-average molecular weight of 2000-10000.

5. The polymeric micelle according to claim 3, wherein the hydrophobic polymeric segment of the block copolymer is bioresorable.

6. The polymeric micelle according to claim 5, wherein the hydrophobic polymer segment of the block copolymer is poly(ester), poly(lactide), poly(lactic acid), or polycaprolactone.

7. The polymeric micelle according to claim 6, wherein the hydrophobic polymer segment of the block copolymer is poly(lactide).

8. The polymeric micelle according to claim 3, wherein the hydrophilic polymeric segment of the graft copolymer is polyacrylate, or a pH-/ionic strength sensitive polymer which is a poly(acrylic acid), poly(methacrylic acid), poly(butenedioic acid), polyhistidine or poly(vinyl imidazole).

9. The polymeric micelle according to claim 3, wherein the hydrophilic polymeric segment of the block copolymer is poly(ester), poly(ethylene glycol), methoxy-poly(ethylene glycol), or poly(2-ethyl-2-oxazoline).

10. The polymeric micelle according to claim 3, wherein said diblock copolymer is methoxy-poly(ethylene glycol)-b-poly(D,L-lactide).

11. The polymeric micelle according to claim 1, wherein the backbone of said graft macromolecule comprises a first repeating unit which is hydrophilic, and the hydrophobic side chains are bound to the first repeating units.

12. The polymeric micelle according to claim 11, wherein the first repeating unit contains a carboxylic group, and the hydrophobic side chains are bioresorable.

13. The polymeric micelle according to claim 12, wherein the backbone of said graft macromolecule is polyacrylate, poly(acrylic acid), poly(methacrylic acid), poly(butenedioic acid), polyhistidine, or poly(vinyl imidazole).

14. The polymeric micelle according to claim 13, wherein the backbone of said graft macromolecule is poly(methacrylic acid).

15. The polymeric micelle according to claim 12, wherein the hydrophobic side chains comprise poly(lactide), poly(lactic acid), or polycaprolactone.

16. The polymeric micelle according to claim 15, wherein the hydrophobic side chains comprise poly(lactide).

17. The polymeric micelle according to claim 11, wherein the backbone of said graft macromolecule further comprises a second repeating unit which is different from the first repeating unit, and the second repeating unit will cause the core collapse in responsive to a temperature change.

18. The polymeric micelle according to claim 17, wherein the second repeating unit of the backbone of said graft macromolecule is derived from a monomer of N-isopropyl acrylamide.

19. The polymeric micelle according to claim 18, wherein the backbone of said graft macromolecule is a copolymer of N-isopropyl acrylamide and methacrylic acid.

20. The polymeric micelle according to claim 1, wherein the polymeric micelle has a diameter of 50-200 nm.

21. The polymeric micelle according to claim 3, wherein said diblock copolymer has a terminal functionality connected to an end of the hydrophilic polymeric segment, and said terminal functionality is a ligand able to be bound to a receptor on a surface of a tumor cell.

22. The polymeric micelle according to claim 21, wherein the ligand is a galactose residue.

23. The polymeric micelle according to claim 3, wherein said diblock copolymer has a terminal functionality connected to an end of the hydrophilic polymeric segment, and said terminal functionality is a fluorescence group.

24. The polymeric micelle according to claim 23, wherein said fluorescence group is a fluorescein isothiocyanate.

25. The polymeric micelle according to claim 3, wherein said diblock copolymer has a terminal functionality connected to an end of the hydrophilic polymeric segment, and said terminal functionality is a dye.

26. The polymeric micelle according to claim 25, wherein said dye is a near infrared dye.

27. The polymeric micelle according to claim 1, wherein said structure comprises a plurality of different block copolymers, and each block copolymer comprising a hydrophobic polymeric segment and a hydrophilic polymeric segment.

28. The polymeric micelle according to claim 27, wherein each of said plurality of different block copolymers is a diblock copolymer comprising a hydrophobic polymeric segment and a hydrophilic polymeric segment.

29. The polymeric micelle according to claim 28, wherein the hydrophobic polymeric segments of the different block copolymers have a same repeating unit.

30. The polymeric micelle according to claim 28, wherein the hydrophilic polymeric segments of the different block copolymers have a same repeating unit.

31. The polymeric micelle according to claim 28, wherein the hydrophilic polymeric segments of the different block copolymers have different repeating units.

32. The polymeric micelle according to claim 28, wherein said plurality of different block copolymers have different terminal functionalities connected to ends of the hydrophilic polymeric segments.

33. The polymeric micelle according to claim 32, wherein one of the terminal functionalities is a ligand able to be bound to a receptor of on a surface of a tumor cell.

34. The polymeric micelle according to claim 33, wherein the ligand is a galactose residue.

35. The polymeric micelle according to claim 32, wherein one of the terminal functionalities is a fluorescence group.

36. The polymeric micelle according to claim 35, wherein said fluorescence group is a fluorescein isothiocyanate.

37. The polymeric micelle according to claim 32, wherein one of the terminal functionalities is a dye.

38. The polymeric micelle according to claim 37, wherein said dye is a near infrared dye.

39. A mixed micelle structure comprising a functional inner core and a hydrophilic outer shell, which is self-assembled from a graft macromolecule and one or more block copolymer.

40. The mixed micelle structure according to claim 39 which is self-assembled from a graft copolymer and two or more diblock copolymers.

41. The mixed micelle structure according to claim 39, which has a size of about 50-200 nm.

42. A process for preparing a polymeric micelle having a core-shell structure, which comprises the following steps:
a) dissolving a graft macromolecule and a block copolymer in an organic solvent, wherein said graft macromolecule comprises a backbone and hydrophobic side chains bound to the backbone, and said block polymer comprises a hydrophobic polymeric segment and a hydrophilic polymeric segment,
b) subjecting the resulting polymer solution from step a) to a dialysis treatment against water to replace the organic solvent in the solution with water.

43. The process according to claim 42 further comprising c) freeze-drying the resulting aqueous solution from step b) to obtain dried polymeric micelle.

44. The process according to claim 42, wherein one or more different block copolymers are dissolved in the organic solvent in step a).

45. The process according to claim 42, wherein a drug is dissolved in the organic solvent together with the graft macromolecule and the block copolymer.
